(19) United States
(12) Patent Application Publication
(10) Pub. No.: US 2008/0125408 A1
(43) Pub. Date: May 29, 2008

(54) PENEM PRODRUG

(75) Inventors: Katherine Elizabeth Brighty, Groton, CT (US); Anthony Marfat, Mystic, CT (US); Dale Gordon McLeod, Ledyard, CT (US); John P. O'Donnell, North Stonington, CT (US); Stephen R. Anderson, Stonington, CT (US)

Correspondence Address: PFIZER INC;Steve T. Zelson, 150 EAST 42ND STREET, 5TH FLOOR - STOP 49, NEW YORK, NY, 10017-5612, US

(73) Assignee: Pfizer Inc

(21) Appl. No.: 11/938,942

(22) Filed: Nov. 13, 2007

Related U.S. Application Data
(60) Provisional application No. 60/978,816, filed on Oct. 10, 2007.
(60) Provisional application No. 60/865,669, filed on Nov. 14, 2006.

(51) Int. Cl. A61K 31/431; C07D 499/897; A61P 31/04

(52) U.S. Cl. 514/195; 540/310

(57) ABSTRACT

The invention relates to prodrugs of sulopenem of formula (I):

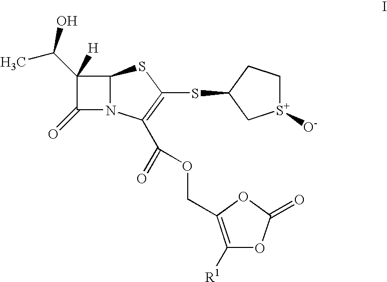

wherein R¹ is —(C₂-C₈)alkyl, or a solvate or hydrate thereof. The invention also relates to the preparation, formulation, and use of the prodrugs of sulopenem to treat a disorder such as an infection in a patient in need thereof.

This application claims the benefit of U.S. Provisional Application No. 60/978,816 filed Oct. 10, 2007 and U.S. Provisional Application No. 60/865,669 filed Nov. 14, 2006, the entire contents of each of the foregoing applications being incorporated herein by reference.

### FIELD OF THE INVENTION

The present invention relates to prodrugs of sulopenem and compositions thereof. The invention also relates to the use of the prodrugs of sulopenem to treat a patient in need thereof.

### BACKGROUND OF THE INVENTION

U.S. Pat. No. 5,013,729 describes sulopenem, which is a broad-spectrum antibiotic that can be named as (5R,6S)-6-[(1R)-1-hydroxyethyl]-7-oxo-3-[(1R,3S)-tetrahydro-1-oxido-3-thienylthio]-4-thia-1-azabicyclo[3.2.0]hept-2-ene-2-carboxylic acid. See also J. Org. Chem., 57, 4352-4361 (1992).

Preclinical and clinical studies have been conducted with sulopenem and certain prodrugs thereof. Sulopenem itself is not appreciably orally bioavailable. The pivaloyloxymethyl ester prodrug (or POM ester) of sulopenem, as a crystalline solid having a suitable melting point and solubility for pharmaceutical practicability, was shown to have less than optimal oral bioavailability in humans at about 500 mg equivalent dose.

Thus, there is a desire for sulopenem prodrugs having high oral bioavailability and physicochemical properties suitable for pharmaceutical use.

### SUMMARY OF THE INVENTION

The present invention relates to prodrugs of sulopenem. In particular, the invention relates to a sulopenem prodrug of formula (I):

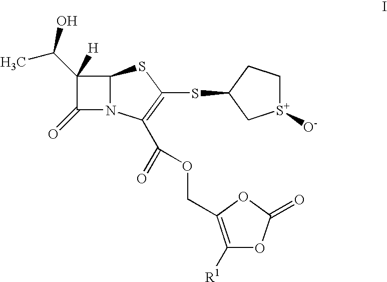

and solvates and hydrates thereof, wherein R¹ is —(C₂-C₈)alkyl.

Unless otherwise specified, the phrase “sulopenem prodrugs of the invention” refers collectively to compounds of formula I, and solvates and hydrates thereof,

In another embodiment, the invention relates to compositions comprising one or more of the sulopenem prodrugs of the invention.

In another embodiment, the invention relates to the use of one or more of the sulopenem prodrugs of the invention to treat an infection.

In one embodiment, the invention relates to methods of making the sulopenem prodrugs of the invention.

In one embodiment, the invention relates to a method of making a sulopenem prodrug of formula I, and solvates and hydrates thereof, comprising allowing a compound of formula (II)

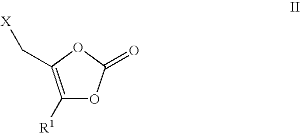

to react with sulopenem (B9):

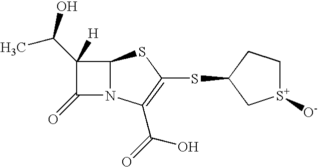

wherein R¹ is —(C₂-C₈)alkyl; and X is a leaving group.

### DETAILED DESCRIPTION OF THE INVENTION

As noted above, in one embodiment, the present invention relates to sulopenem prodrugs as described above.

As used herein, the term “(C₂-C₈)alkyl” refers to linear or branched (e.g., ethyl, n-propyl, isopropyl, n-butyl, iso-butyl, secondary-butyl, tertiary-butyl, n-pentyl, n-hexyl, n-heptyl and n-octyl) radicals of 2 to 8 carbon atoms.

In one embodiment, the sulopenem prodrug is a compound of formula I wherein R¹ is —CH₂CH₃ (compound 1).

In another embodiment, the sulopenem prodrug is a compound of formula I wherein R¹ is —CH₂CH₃CH₃ (compound 2).

In another embodiment, the sulopenem prodrug is a compound of formula I wherein R¹ is —CH₂CH(CH₃)₂ (compound 3).

The sulopenem prodrugs of the invention may exist in unsolvated and solvated forms. Thus, it will be understood that the compounds of the invention also include hydrate and solvate forms as discussed below.

The term “solvate” is used herein to describe a noncovalent or easily reversible combination between solvent and solute, or dispersion means and disperse phase. It will be understood that the solvate can be in the form of a solid, slurry (e.g., a suspension or dispersion), or solution. Non-limiting examples of solvents include ethanol, methanol, propanol, acetonitrile, dimethyl ether, diethyl ether, tetrahydrofuran, methylene chloride, and water. The term ‘hydrate’ is employed when said solvent is water.

A currently accepted classification system for organic hydrates is one that defines isolated site, or channel hydrates—see Polymorphism in Pharmaceutical Solids by K. R. Morris (Ed. H. G. Brittain, Marcel Dekker, 1995). Isolated site hydrates are ones in which the water molecules are isolated from direct contact with each other by intervening organic molecules. In channel hydrates, the water molecules lie in lattice channels where they are next to other water molecules.

When the solvent or water is tightly bound, the complex will have a well-defined stoichiometry independent of humidity. When, however, the solvent or water is weakly bound, as in channel solvates and hygroscopic compounds, the water/solvent content will be dependent on humidity and drying conditions. In such cases, non-stoichiometry will be the norm.

The present invention also includes isotopically-labeled compounds, which are identical to those of the sulopenem prodrugs of the invention, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, and sulfur, such as, but not limited to, ²H, ³H, ¹³C, ¹⁴C, ¹⁵N, ¹⁸O, ¹⁷O, and ³⁵S, respectively. The sulopenem prodrugs of the invention containing the aforementioned isotopes and/or other isotopes of these atoms are within the scope of this invention. Certain isotopically-labeled sulopenem prodrugs of the invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with isotopes such as deuterium, i.e., ²H, can afford certain therapeutic advantages resulting from greater metabolic stability, for example increased in vivo half-life or reduced dosage requirements and, hence, may be preferred in some circumstances. Isotopically-labeled prodrugs of the invention can generally be prepared by carrying out the procedures disclosed in the Schemes and/or in the Examples and described below, by substituting a readily available isotopically-labeled reagent for a non-isotopically-labeled reagent.

The sulopenem prodrugs of the invention may exhibit polymorphism. Polymorphs of the sulopenem prodrugs of the invention may be prepared by crystallization of a prodrug of the present invention under various conditions. For example, there may be employed various solvents (including water) or different solvent mixtures for recrystallization; crystallization at different temperatures; various modes of cooling ranging from very fast to very slow cooling during crystallization. Polymorphs may also be obtained by heating or melting a prodrug followed by gradual or fast cooling. The presence of polymorphs may be determined by solid probe NMR spectroscopy, IR spectroscopy, differential scanning calorimetry, powder X-ray diffraction or other such techniques.

The invention also relates to compositions of the invention which comprise any combination of one or more sulopenem prodrugs of the invention and at least one additional ingredient (hereinafter “the compositions of the invention”). In one embodiment, the composition of the invention comprises a therapeutically effective amount of the sulopenem prodrug of the invention.

Non-limiting examples of the at least one additional ingredient include impurities (e.g., intermediates present in the unrefined sulopenem prodrugs of the invention), active or pharmaceutical agents as discussed below (e.g., another antibacterial agent), pharmaceutically acceptable excipients, or one or more solvents (e.g., a pharmaceutically acceptable carrier as discussed herein).

The term “solvent” as it relates to the compositions of the invention includes organic solvents (e.g., methanol, ethanol, isopropanol, ethyl acetate, methylene chloride, and tetrahydrofuran) and water. The one or more solvents may be present in a non-stoichiometric amount, e.g., as a trace impurity, or in sufficient excess to dissolve the compound of the invention. Alternatively, the one or more solvents may be present in a stoichiometric amount, e.g., 0.5:1, 1:1, or 2:1 molar ratio, based on the amount of compound of the invention.

In one embodiment, the at least one additional ingredient that is present in the composition of the invention is an organic solvent.

In another embodiment, the at least one additional ingredient that is present in the composition of the invention is water.

In one embodiment, the at least one additional ingredient that is present in the composition of the invention is a pharmaceutically acceptable carrier.

In another embodiment, the at least one additional ingredient that is present in the composition of the invention is a pharmaceutically acceptable excipient as discussed below.

In one embodiment, the composition of the invention is a solution.

In another embodiment, the composition of the invention is a suspension.

In another embodiment, the composition of the invention is a solid.

Compositions of the invention that are suitable for administration to a patient in need thereof (e.g., a human) are also referred to herein as “pharmaceutical compositions of the invention.”

The pharmaceutical composition may be in any form suitable for administration to a patient. For example, the pharmaceutical composition may be in a form suitable for oral administration such as a tablet, capsule, pill, powder, sustained release formulations, solution, and suspension; for parenteral injection as a sterile solution, suspension or emulsion; for topical administration as an ointment or cream; or for rectal administration as a suppository. The pharmaceutical composition may be in unit dosage forms suitable for single administration of precise dosages.

Exemplary parenteral administration forms include solutions or suspensions of active compounds in sterile aqueous solutions, for example, aqueous propylene glycol or dextrose solutions. Such dosage forms can be suitably buffered, if desired.

In one embodiment, the pharmaceutical composition of the invention may be in the form of an oral dosage form. Non-limiting examples of oral dosage forms include such as, e.g., chewable tablets, capsules, pills, lozenges, troches, sachets, powders, syrups, elixirs, solutions and suspensions, and the like, in accordance with standard pharmaceutical practice.

In another embodiment, the pharmaceutical composition of the invention can also be delivered directly to a patient's gastrointestinal tract through a nasogastric tube.

The sulopenem prodrugs of the invention will be present in the pharmaceutical composition of the invention in an amount sufficient to provide the desired dosage amount in the range described herein. The proportional ratio of prodrug to excipients will naturally depend on the chemical nature, solubility and stability of the active ingredients, as well as the dosage form contemplated. Typically, pharmaceutical compositions of the present invention can contain about 20% to about 95% of prodrug by weight.

In one embodiment, the amount of the sulopenem prodrug present in the pharmaceutical composition of the invention is from about 200 mg to about 4000 mg of sulopenem prodrug.

In another embodiment, the amount of the sulopenem prodrug present in the composition of the invention is from about 200 mg to about 3000 mg of sulopenem prodrug.

In another, the amount of the sulopenem prodrug present in the pharmaceutical composition of the invention is from about 200 mg to about 2000 mg of sulopenem prodrug.

In another embodiment, the amount of the sulopenem prodrug present in the composition of the invention is from about 400 mg to about 4000 mg of sulopenem prodrug.

In another, the amount of the sulopenem prodrug present in the pharmaceutical composition of the invention is from about 400 mg to about 3000 mg of sulopenem prodrug.

In another, the amount of the sulopenem prodrug present in the pharmaceutical composition of the invention is from about 400 mg to about 2000 mg of sulopenem prodrug.

In one embodiment, the sulopenem prodrug used in the composition of the invention is sulopenem prodrug 1.

In another embodiment, the sulopenem prodrug used in the composition of the invention is sulopenem prodrug 2.

In another embodiment, the sulopenem prodrug used in the composition of the invention is sulopenem prodrug 3.

Techniques for formulation and administration of the prodrugs of the instant invention can be found in Remington: the Science and Practice of Pharmacy, 19th ed., Mack Pub. Co., Easton, Pa. (1995).

The term “excipient” means an inert material that is combined with the sulopenem prodrug to produce a pharmaceutical composition or oral drug dosage form. The term “pharmaceutically acceptable excipient” means that the excipient must be compatible with other ingredients of the composition, and not deleterious to the recipient thereof. The pharmaceutically acceptable excipients are chosen on the basis of the intended dosage form.

The tablets, pills, capsules, and the like may contain excipients selected from binders such as polyvinylpyrrolidone, hydroxypropylmethyl cellulose (HPMC), hydroxypropylcellulose (HPC), sucrose, gelatin, acacia, gum tragacanth, or corn starch; fillers such as microcrystalline cellulose, lactose, sodium citrate, calcium carbonate, dibasic calcium phosphate, glycine and starch; disintegrants such as corn starch, potato starch, alginic acid, sodium starch glycolate, croscarmellose sodium and certain complex silicates; lubricants such as magnesium stearate, sodium lauryl sulfate and talc; and sweeteners such as sucrose lactose or saccharin. When a dosage unit form is a capsule, it may contain, in addition to materials of the above type, a liquid carrier such as a fatty oil. Various other materials may be present as coatings or to modify the physical form of the dosage unit. For instance, tablets may be coated with shellac, sugar or both.

In the case of pediatric oral suspensions and sachets, these excipients may comprise suspending aids such as xantham gum or hydroxypropylmethylcellulose, glidants such as colloidal silica, diluents and bulking agents such as silicon dioxide, flavors such as bubble gum, orange, banana, raspberry and golden syrup or mixtures thereof, sweeteners such as aspartame or sugar, and stabilizers such as succinic acid. Powder or granular formulations, such as pediatric suspension formulations and sachets, may be manufactured using techniques which are generally conventional in the field of manufacture of pharmaceutical formulations and in the manufacture of dry formulations for reconstitution into such suspensions. For example a suitable technique is that of mixing dry powdered or granulated ingredients.

As noted above, the present invention also relates to methods of making the sulopenem prodrugs of the invention comprising allowing the compound of formula II to react with sulopenem (B9). The reaction is carried out under conditions sufficient to form the sulopenem prodrugs of the invention. Typically, the reaction between the compound of formula I and sulopenem is carried out in an organic solvent and in the presence of base. Non-limiting examples of suitable organic solvents include ketones such as acetone and methylethylketone. Non-limiting examples of suitable bases include amines such as dialkyl- and trialkylamines. In one embodiment for making the sulopenem prodrugs of the invention, the base is a trialkylamine; in another embodiment, the base is diisopropylethylamine.

The reaction for making the sulopenem prodrugs is carried out for a time and at a temperature sufficient to form the sulopenem prodrug of the invention. For example, a typical time for carrying out the reaction is from about 0.25 hours to about 72 hours. The temperature for carrying out the reaction can be from about just above the freezing point of the solvent to the reflux temperature of the solvent. Typically, the temperature for carrying out the reaction is from about 0° C. to about 100° C.; more typically from about 15° C. to about 45° C.

In one embodiment for making the sulopenem prodrugs of the invention, the compound of formula II is a compound where R¹ is —CH₂CH₃; in another embodiment; R¹ is —CH₂CH₃CH₃; and in another embodiment, R¹ is —CH₂CH(CH₃)₂.

In one embodiment for making the sulopenem prodrugs of the invention, the compound of formula II is a compound where X is selected from the group consisting of -halo, -methanesulfonate (mesylate), -trifluoromethanesulfonate (triflate), and -p-toluenesulfonate (tosylate).

In one embodiment for making the sulopenem prodrugs of the invention, the compound of formula II is a compound where X is bromo.

The sulopenem prodrugs of the invention and the compounds of formula II can be prepared in a manner similar to that described for the preparation of 3 described in the Examples section and described in U.S. Pat. No. 5,013,729, the entire content of which is incorporated herein by reference.

Other methods useful for making the sulopenem prodrugs of the invention can be found in U.S. Pat. Nos. 3,951,954, 4,234,579, 4,287,181, 4,452,796, 4,342,693, 4,348,264, 4,416,891, 4,457,924, and 5,013,729, entire contents of each of the foregoing being incorporated herein by reference. These patents describe methods involving the reaction of the free acid of sulopenem or cationic salts of sulopenem. Preferably, cationic salts of sulopenem are tetra-n-butyl ammonium or diisopropylethyl ammonium salts, which are prepared by reacting the sulopenem free acid with tetrabutyl ammonium hydrogen sulfate (n-Bu)₄N⁺HSO₄⁻ or diisopropylethyl-amine (DIEA) respectively. The resulting sulopenem cationic salt can then be reacted with the appropriate alkyl halide to form the desired prodrug.

In some embodiments, the oral bioavailability, preferably human oral bioavailability, (fraction absorbed, % F) of the sulopenem prodrugs of the invention is at least about 25%, 30%, 40%, 50%, 60%, or greater. Oral bioavailability can be predicted variously by factors including one or more of (1) GI tract stability, (2) Caco-2 permeability, (3) efficiency of conversion to sulopenem, and (4) high solubility. Accordingly, the sulopenem prodrugs of the invention were evaluated, as detailed below, for stability in human intestinal juice (HIJ), efficiency of conversion to sulopenem in human liver homogenate, whole blood conversion and solubility as measured in phosphate buffer pH 5.0.

Sulopenem prodrugs of the invention were tested according to the following general procedures and the results are shown in Table 2.

### Liver S9 and Whole Blood Conversion Efficiency Studies:

Liver S9 was prepared fresh from liver chunks stored at −70° C. for each analysis completed. Approximately 5 g of frozen liver tissue was homogenized to uniformity in 15 mL of ice cold 100 mM potassium phosphate (pH 7.4) buffer. The homogenate was then centrifuged at 9000 g for 20 minutes at 5° C. to isolate the S9 supernatant fraction. Each incubation was run at a 1:10 dilution of the S9 supernatant in 100 mM potassium phosphate (pH 7.4) buffer. Reactions (1 mL) were initiated by the addition of substrate (50 μM final) at 37° C.

Stability and conversion efficiency were assessed in 1 mL incubations with heparinized whole blood at 37° C. For both S9 homogenates and whole blood incubations, aliquots (75 μL) were obtained at 0, 0.5, 1, 2, 3, 5, 10, and 20 minutes and quenched in 150 μL of 80/20 acetonitrile/100 mM ammonium acetate pH 4.5 containing an internal standard (ampicillin, 5 μg/mL). Samples were centrifuged at 3000 g for 10 minutes and the supernatants transferred to injection vials. First order degradation of the prodrug was monitored by LC/MS/MS as described below. Conversion to sulopenem was expressed as a percentage of the molar equivalent (50 μM) in a fortified sample. The results of the S9 and whole blood stability and conversion studies are shown in Table 2, where single values represent an average of two duplicate determinations. The results (Table 2) show that the liver S9 conversion to sulopenem is about 67% or greater.

### Human Intestinal Juice (HIJ) Studies:

In the HIJ experiments, HIJ from 4 individual subjects (1 mL each) was pooled with 1 mL of 600 mM potassium phosphate buffer pH 7.4. Aliquots of 300 μL×6 of the buffered human intestinal juice were incubated at 37° C. following fortification of substrate at concentrations of 300, 100, 30, 10, 3, and 1 μM. Two prodrug compounds could be run at the same time. Samples of 35 μL were taken at 0, 0.5, 1, 2, 10, and 20 minutes and quenched with 70 μL of 80/20 acetonitrile/100 mM ammonium acetate pH 4.5 containing an internal standard (ampicillin, 5 μg/mL). Samples were centrifuged at 3000 g for 10 minutes and the supernatants transferred to injection vials. First order degradation of the prodrug was monitored by LC/MS/MS as described below. The percentage of prodrug remaining versus time at each concentration was fitted to a first order decay function to determine the substrate depletion rate constant or k_{dep}. A linear-log plot of k_{dep} versus concentration could be fitted with the following equation where:

$${k}_{dep} = {k}_{dep [ S ] = 0} \cdot ( 1 - \frac{[ S ]}{[ S ] + {K}_{m}} )$$

The value of k_{dep} at an infinitesimally low substrate concentration (where k_{dep}˜k_{dep[s]=0}) represents the maximum consumption rate or intrinsic clearance of the system and the Kₘ is the concentration at which half of the maximal velocity of the system is achieved. In Michaelis-Menton terms, intrinsic clearance CLᵢₙₜ represents the ratio of Vₘₐₓ/Kₘ when [S] is well below the Kₘ. Substrate units for these studies were reported in μM and intrinsic clearance (CIᵢₙₜ) in mL/min. Results of these studies are shown in Table 2.

### Caco-2 Permeability Studies:

The extent of intestinal absorption is directly proportional to the rate, or permeation, at which a compound crosses the intestinal epithelium from gut lumen to blood (when solubility is not limiting). Accurately determining this permeation can allow predictions of the extent of intestinal absorption to be made. The Caco-2 model is an in vivo tissue culture model of human intestinal epithelium that can be used to study a compound's apparent permeation, or permeability (Pₐₚₚ), across intestinal epithelium. This model forms confluent monolayers and spontaneously differentiates with well defined apical (A) and basolateral (B) domains separated by intercellular junctional complexes. Each domain contains distinct biochemical features analogous to those present in the intestinal epithelium (e.g. membrane composition, transporters and enzymes). The luminal side of the intestine is modeled by the A compartment and the serosal side of the intestine is modeled by the B compartment. The Caco-2 model is cultured in a bicameral format allowing the appearance (rather than disappearance, as is typically measured in vivo or in situ) of a compound from a donor compartment (compartment to which experimental solution is applied) into a receiver compartment (containing appropriate transport buffer) to be measured. Thus with this model, a true apparent permeability (Pₐₚₚ) across polarized epithelium can be determined. This Pₐₚₚ will be principally determined by the combination of the compound's passive permeability and any potential cellular biochemical activity that may affect compound transport across the monolayer. By performing assays using this model, which is similar in architecture and biochemical activity to the intestinal epithelium, insight may be gained into the disposition (with regards to permeation and the mechanisms that determine permeation) of the compound across human intestinal epithelium. Because the cells contain various esterases capable of hydrolyzing the prodrug, quantification of both prodrug and sulopenem (B9) is an important aspect in assessing the mass transfer and recovery within the system.

An A→B assay is preformed to assess the test compound Pₐₚₚ in the absorptive transport direction (luminal to serosal; P_{app,A→B}). In an A→B assay, the compound is placed in the A compartment and the appearance of the compound is monitored in the B compartment as a function of time.

Materials: Caco-2 cells were obtained from American Type Culture Collection (Rockville, Md.). Cell culture medium (Dulbecco's Modified Eagles Medium with 20% fetal bovine serum, 1% Non-essential amino acids, 1% Glutamax-1 and 0.08% gentamycin) and transport buffers (Hank's balanced salt solution with 25 mM D-glucose monohydrate, 1.25 mM CaCl₂ and 0.5 mM MgCl₂) A (transport buffer with 20 mM 2-[N-Morpholino]ethanesulfonic acid (MES); pH 6.5) and B (transport buffer with 20 mM N-hydroxyethylpiperazine-N′-2-ethanesulfonate (HEPES); pH 7.4) were obtained from Invitrogen (Gibco Laboratories, Grand Island, N.Y.). HTS 24-Multiwell insert system cell culture plates (polyethylene terephthalate (PET) membrane, 0.28 cm² growth area, 1 μm pore size) were obtained from BD Falcon (Bedford, Mass.).

Caco-2 Cell Culture: Caco-2 cells were cultured at 37° C. with cell culture medium in an atmosphere of 10% CO₂ and 90% relative humidity in a Nuaire Incubator (Plymouth, Minn.). The cells were passaged upon reaching approximately 75-85% confluence from T-flasks using 0.05% trypsin-EDTA (Invitrogen, Gibco Laboratories, Grand Island, N.Y.). Caco-2 cells were seeded onto each PET membrane of the HTS 24-Multiwell insert using 500 μL of a 100,000 cells/mL cell suspension in cell culture medium. To the feeding tray was added 25 mL of cell culture medium. The cell culture medium was changed bi-weekly and was changed 24 h prior to experimentation. Caco-2 cells monolayers were used for experimentation 26-days post seeding.

Assay Protocol: The Caco-2 cell culture media was removed from both compartments and 300 μL of transport buffer A was placed in A compartment and 500 μL of transport buffer B was placed in B compartment. The monolayers were incubated for 1 hour at 37° C. in an Orbital EnvironShaker (Lab-Line; Dubuque, Iowa) rotating at 100 rpm. After 1 hour, the buffers were removed from both compartments.

To conduct an A→B assay, 300 μl of a 100 μM prodrug solution containing lucifer yellow (0.1 mg/mL) as a monolayer integrity standard in transport buffer A was added to the A compartment and 500 μl of transport buffer B was added to the B compartment (n=3 monolayers per condition). Monolayers were incubated for 1 hour in the orbital shaker at 100 rpm and 37° C. The entire transwell insert was transferred to a new 24-well plate containing fresh transport buffer B (pre-incubated at 37° C.) and placed back in the orbital shaker for another h at 100 rpm and 37° C. The assay samples collected from compartment B (well of 24-well plate) following 1 hour were retained for sample analysis. At the end of the second hour, samples from both compartments were removed and saved for analysis. Sample from B compartment was the assay sample for the second hour and sample from A compartment was collected for determination of recovery. All samples obtained from buffers A and B were quantified for both the prodrugs of sulopenem and sulopenem (B9) by LC/MS/MS methods. Total mass for each compartment was calculated based upon contributions from both intact prodrug and sulopenem. Lucifer yellow content was determined by using a Wallac Victor 11 Fluorescence Detector (Turku, Finland) at excitation wavelength of 430 nm and emission wavelength of 535 nm.

Data Analysis: Pₐₚₚ (×10⁻⁶ cm/sec) was calculated using equation 1:

$${P}_{app} = \frac{1}{Area * {C}_{D} ( 0 )} * \frac{d {M}_{r}}{d t} \qquad Eq . 1$$

where Area is the surface area of the cell monolayer (0.3 cm²), C_{D}(0) is the initial concentration of compound applied to the donor chamber, t is time, Mᵣ is the mass of compound in the receiver compartment, and dMᵣ/dt is the flux of the compound across the cell monolayer.

Lucifer yellow flux was determined using equation 2:

$$\% flux = ( \frac{{C}_{R}}{{C}_{D ( 0 )}} ) * 100 \qquad Eq . 2$$

where C_{R} is the concentration of lucifer yellow in the receiver compartment after 1 hour and C_{D}(0) is the initial concentration of lucifer yellow applied to the donor chamber. Monolayers with lucifer yellow flux<3%/h were determined to be intact. Data from monolayers with lucifer yellow flux>3%/h was not included.

Compound recovery was determined using equation 3:

$$\% Recovery = ( \frac{{C}_{R ( 1 )} + {C}_{R ( 2 )} + {C}_{D ( 2 )} + {C}_{M}}{{C}_{D ( 0 )}} ) * 100 \qquad Eq . 3$$

where C_{R(1)} is the concentration of compound in the receiver compartment after 1 h, C_{R(2)} is the concentration of compound in the receiver compartment after 2 h, C_{D(2)} is the concentration of compound in the donor compartment after 2 h, C_{M} is the concentration of drug remaining in the monolayer, and C_{D(0)} is the initial concentration of compound applied to the donor compartment. Recovery was typically 90% or greater.

### Solubility:

Equilibrium solubility was determined in 25 mM phosphate buffer (pH 5) at ambient temperature. Vials containing excess prodrug in phosphate buffer were rotated for up to 48 hours. After the equilibrium period, samples were pulled, filtered through a 0.45 u Gelman Acrodisc Nylon syringe filter and analyzed for drug concentration using HPLC. The HPLC conditions were: Column: C18, SymmetryShield RP, Waters, 4.6×150 mm, 3.5 micron; Mobile phase A: Acetonitrile; Mobile phase B: 0.1% TFA in water; Flow rate: 1 mL/min; Run Time: 30 min; Inj. Vol 20 uL; Detection: 350 nm; RT=16 min; Dissolving solvent: Acetonitrile/water (50:50).

Gradient used:

| Time | % A | % B |
|---|---|---|
| 0 min | 5 | 95 |
| 25 min | 95 | 5 |
| 27 min | 5 | 95 |
| 30 min | 5 | 95 |

### Melting Points:

Melting points were determined on a MEL-TEMP 3.0 capillary melting point apparatus and were uncorrected. Results are shown in Table 2.

### Methods of Use:

The sulopenem prodrugs of the invention are useful for treating a patient suffering from a disorder such as, e.g., a bacterial infection.

As used herein the term “patient” refers to a mammal such as, e.g., a human, dog, cat, horse, pig, cow, and the like. In one embodiment, the patient is a human.

Bacterial infections amenable to treatment by the sulopenem prodrugs of the invention, pharmaceutical compositions and methods of the present invention include those caused by a broad range of pathogens, such as, but not limited to, Staphylococcus aureus, Staphylococcus saprophyticus, Alloiococcus otitidis, Streptococcus pyogenes, Streptococcus agalactiae, Viridans Streptococcus, Streptococcus pneumoniae penicillin-susceptible, Streptococcus pneumoniae penicillin-resistant, Streptococcus pneumoniae levofloxacin-resistant, Listeria monocytogenes, Citrobacter diversus, Citrobacter freundii, Enterobacter aerogenes, Enterobacter cloacae, Escherichia coli, Klebsiella oxytoca, Klebsiella pneumoniae, Klebsiella pneumoniae (including those encoding extended-spectrum β-lactamases (hereinafter “ESBLs”), Morganella morgani, Proteus mirabilis, Salmonella/Shigella, Haemophilus influenzae β-lactamase negative, Haemophilus influenzae β-lactamase positive, Moraxella catarrhalis β-lactamase-negative, Moraxella catanhalis β-positive, Legionella pneumophila, Neisseria meningitidis, Bacteroides fragilis, Clostridium perfringens, Prevotella spp. and members of the Enterobacteriaceae that express ESBLs and AmpC-type beta-lactamases that confer resistance to currently available cephalosporins, cephamycins and beta-lactam/beta-lactamase inhibitor combinations. Sulopenem, and the prodrugs thereof, are not active against Pseudomonas aeruginosa, Acinetobacter spp., Stenotrophomonas maltophilia, multi-resistant enterococci and methicillin-resistant staphylococci.

In one embodiment, the prodrugs of the invention are active against gram-positive bacteria.

In another embodiment, the prodrugs of the invention are active against gram-negative bacteria except for Pseudomonas aeruginosa, Acinetobacter spp. and Stenotrophomonas maltophilia.

The in vitro activity of sulopenem (B9) (the parent acid) based on minimum inhibitory concentration (MIC) was evaluated against pathogens involved in 40 community and hospital infections, as summarized in Table 1.

TABLE 1
MIC₉₀ values (ug/mL) for Sulopenem

| | |
|---|---|
| Staphylococcus aureus oxacillin-S | 0.125 |
| Staphylococcus saprophyticus | 0.5 |
| Alloiococcus otitidis | 1 |
| Streptococcus pyogenes (Group A) | 0.03 |
| Streptococcus agalactiae (Group B) | 0.125 |
| Streptococcus bovis (Group D) | 0.06 |
| Viridans Streptococcus group | 0.25 |
| Streptococcus pneumoniae penicillin-susceptible | 0.03 |
| Streptococcus pneumonia penicillin-intermediate | 0.25 |
| Streptococcus pneumoniae penicillin-resistant | 1 |
| Streptococcus pneumoniae levofloxacin-resistant | 0.5 |
| Listeria monocytogenes | 0.125 |
| Corynebacterium spp (not C. jeikeium) | 2 |
| Citrobacter diversus | 0.06 |
| Citrobacter freundii | 0.25 |
| Enterobacter aerogenes | 0.5 |
| Enterobacter cloacae | 1 |
| Escherichia coli | 0.06 |
| Klebsiella oxytoca | 0.125 |
| Klebsiella pneumoniae | 0.125 |
| Klebsiella pneumoniae ESBL+ | 0.25 |
| Morganella morganii | 2 |
| Proteus mirabilis | 0.5 |
| Salmonella/Shigella | 0.125 |
| Haemophilus influenzae β-lactamase− | 0.25 |
| Haemophilus influenzae β-lactamase+ | 0.5 |
| Moraxella catarrhalis β-lactamase− | 0.03 |
| Moraxella catarrhalis β-lactamase+ | 0.125 |
| Legionella pneumophila | 0.06 |
| Neisseria meningitides | 0.06 |
| Bacteroides fragilis | 0.5 |
| Clostridium perfringens | 0.06 |
| Prevotella spp | 0.125 |

The sulopenem prodrugs of the invention may, in one embodiment, be used to treat a variety of hospital and community acquired infections such as respiratory tract, surgical, central nervous system, gastrointestinal, genitourinary, gynecological, skin & soft tissue, and ocular infections and community acquired pneumonia in humans (hereinafter “the infections”).

In one embodiment, the infection is selected from the group consisting of acute exacerbation of chronic bronchitis, sinusitis, otitis media, brain abscess, pharyngitis, meningitis, uncomplicated/complicated urinary tract infections, pyelonephritis, hospital-acquired pneumonia, community-acquired pneumonia, surgical prophylaxis, uncomplicated/complicated skin and skin structure infections, intra-abdominal infections, prostatitis, obstetric and gynecological infections, bone and joint infections, diabetic foot, and bacteremia.

In another embodiment, the infection is selected from the group consisting of complicated and uncomplicated urinary tract infections, complicated skin and skin structure infections, diabetic foot infections, community-acquired pneumonia, hospital-acquired pneumonia, intra-abdominal infections, and obstetric and gynecological infections.

The minimum amount of the sulopenem prodrug of the invention to be administered is a therapeutically effective amount. The term “therapeutically effective amount” means the amount of prodrug which prevents the onset of, alleviates the symptoms of, stops the progression of, and/or eliminates a bacterial infection in a mammal, e.g., a human.

The maximum amount of the sulopenem prodrug of the invention administered is that amount which is toxicologically acceptable; in one preferred embodiment, the amount of the sulopenem prodrug of the invention administered is the amount which will maintain the plasma antibiotic concentration of sulopenem above the MICs of the infecting pathogens for at least about 30%, preferably at least about 40%, of the interval between doses (dose interval). In a more preferred embodiment, the amount of the sulopenem prodrug of the invention administered is the amount which will maintain the plasma antibiotic concentration of sulopenem above the MICs of the infecting pathogens for at least 40% of the dose interval.

Typically, an effective daily dose (i.e., total dosage over about 24 hours) of the sulopenem prodrug of the invention for adults is about 400 mg to about 6000 mg of sulopenem prodrug; in another embodiment, an effective daily dose is about 800 mg to about 6000 mg of sulopenem prodrug; and in another embodiment, an effective daily dose is about 800 mg to about 4000 mg of sulopenem prodrug. This daily dose is administered over a period of about one week to about two weeks. In some cases, it may be necessary to use dosages outside these limits.

The amount of the effective daily dose may be adjusted to account for body mass. In one embodiment, the effective daily dose (i.e., total dosage over about 24 hours) of the sulopenem prodrug of the invention for adults is about 5 mg to about 85 mg of sulopenem prodrug per kilogram of body weight; in another embodiment, an effective daily dose is about 10 mg to about 85 mg of sulopenem prodrug per kilogram of body weight; and in another embodiment, an effective daily dose is about 10 mg to about 60 mg of sulopenem prodrug per kilogram of body weigh. This daily dose is administered over a period of about one week to about two weeks. In some cases, it may be necessary to use dosages outside these limits.

A daily dosage of the sulopenem prodrug of the invention is usually administered from 2 to 4 times daily in equal doses.

In one embodiment, a single dose of sulopenem prodrug is administered per day (i.e., over about 24 hours) (i.e., QD); in another embodiment, two doses of sulopenem prodrug are administered per day (i.e., BID); in another embodiment, three doses of sulopenem prodrug are administered per day (i.e., TID); and in another embodiment, four doses of sulopenem prodrug are administered per day (i.e., QID).

In one embodiment, the effective dose of the sulopenem prodrug of the invention is administered BID in about 12 hour intervals.

In another embodiment, the effective dose of the sulopenem prodrug of the invention is administered TID in about 8 hour intervals.

In another embodiment, the effective dose of the sulopenem prodrug of the invention for is administered QID in about 6 hour intervals.

In one embodiment, an effective dose of the sulopenem prodrug of the invention is about 400 mg to about 3000 mg which is administered BID in about 12 hour intervals.

In another embodiment, an effective dose of the sulopenem prodrug of the invention is about 400 mg to about 2000 mg which is administered TID in about 8 hour intervals.

In another embodiment, an effective dose of the sulopenem prodrug of the invention is about 400 mg to about 1500 mg which is administered QID in about 6 hour intervals.

The sulopenem prodrugs of the invention readily hydrolyze in vivo after absorption to form sulopenem, which is the antibacterially-active form. The sulopenem prodrugs of the invention have a high oral bioavailability in humans which is sufficient to achieve a drug exposure above a desired level, e.g., 1 ug/mL for at least 40% of the dosing interval to effectively treat bacterial infections.

In one embodiment, the sulopenem prodrug used in the treatment of an infection is sulopenem prodrug 1.

In another embodiment, the sulopenem prodrug used in the treatment of an infection is sulopenem prodrug 2.

In another embodiment, the sulopenem prodrug used in the treatment of an infection is sulopenem prodrug 3.

Oral administration is preferred.

The sulopenem prodrugs of the invention may be administered in combination with one or more additional medicinal or pharmaceutical agents (“the additional active agent”). Such use of sulopenem prodrugs of the invention in combination with an additional active agent may be for simultaneous, separate or sequential use.

In one embodiment, the additional active agent is an antibacterial agent. Non-limiting examples of useful antibacterial agents include:

aminoglycosides such as streptomycin, gentamycin, kanamycin or amikacin;

ansamycins such as rifamycin;

β-lactams such as penicillins (e.g., amoxicillin and ampicillin), cephalosporins (e.g., cefipime, cefditoren pivoxil (Spectracef®), cephalothin, cefaclor or cefixime;

β-lactamase inhibitors and β-lactam/β-lactamase inhibitor combinations such as sulbactam, clavulanic acid, tazobactam and piperacillin-tazobactam (Zosyn®);

carbapenems such as ertapenem (Invanz®), imipenem-cilastatin (Primaxin®) and meropenem (Merrem®);

dihydrofolate reductase inhibitors such as iclaprim;

glycopeptides such as vancomycin (Vancocin®), dalbavancin (Pfizer), oritavancin (Targenta Therapeutics), telavancin (Theravance), ramoplanin (Pfizer and Oscient), teicoplanin (Targocid®);

ketolides such as telithromycin (Ketek®);

lipopeptides such as daptomycin (Cubicin®);

lincosamides such as clindamycin and lincomycin;

LpxC inhibitors such as those disclosed in WO2007069020 and WO200407444;

macrolides such as azithromycin, erythromycin, or clarithromycin;

oxazolidinones such as linezolid (Zyvox®), ranbezolid (RBX 7644), DA 7867, AZD-2563; the compounds disclosed in U.S. Pat. No. 7,141,588; and the compounds disclosed in U.S. Patent Application Publication Nos. 20040176610 and 20060030609:

polymyxins such as polymyxin B sulfate and colistin;

quinolones and fluoroquinolones such as norfloxacin, ciprofloxacin, levofloxacin (Levaquin®), gemifloxacin (Factive®), moxifloxacin (Avelox®), nalidixic acid or enoxacin;

phenylpropanoids such as chloramphenicol;

phosphonates such as fosfomycin;

sulfonamides such as sulfapyridine; and

tetracyclines such as chlortetracycline, doxycycline, tigecycline (Tygacil®).

Other non-limiting examples of additional antibacterial agents can be found in Chemical Reviews 105(2): 391-394 (2005); and Bush et al., Current Opinion in Microbiology 7:466-476 (2004); the entire contents of each of the foregoing references being incorporated herein in their entirety.

In one embodiment, the additional active agent is probenecid. Without being limited by theory, it is believed that administration of the sulopenem prodrugs of the invention in combination with probenicid increases the half-life of the active form of the prodrug.

In one embodiment, the one or more additional active agents, when used, are administered prior to administration of the sulopenem prodrugs of the invention. In another embodiment, the one or more additional active agents, when used, are administered after administration of the sulopenem prodrugs of the invention. In another embodiment, the one or more additional active agents, when used, are administered at about the same time as administration of the sulopenem prodrugs of the invention.

The additional active agent may be administered by any route useful to administer said additional active agent.

In one embodiment, the one or more additional active agents are present in the pharmaceutical composition of the invention. Accordingly, in another embodiment, the invention relates to a method of treating a patient with a pharmaceutical composition of the invention further comprising one or more additional active agents.

The examples and preparations provided below further illustrate and exemplify the compounds of the present invention and methods of preparing such compounds. It is to be understood that the scope of the present invention is not limited in any way by the scope of the following examples and preparations.

All patents, applications, publications, test methods, literature, and other materials cited herein are hereby incorporated herein by reference in their entireties.

### EXAMPLES

The present invention will be further illustrated by means of the following nonlimiting examples.

### Example 1

Preparation of (5-(3-methylpropyl)-2-oxo-1,3-dioxol-4-yl)methyl (5R,6S)-6-[(1R)-1-hydroxyethyl]-7-oxo-3-[[(1R,3S)-tetrahydro-1-oxido-3-thienyl]thio]-4-thia-1-azabicyclo[3.2.0]hept-2-ene-2-carboxylate (3): Compound 3 was prepared according to the process depicted in Scheme 1 and described in detail below:

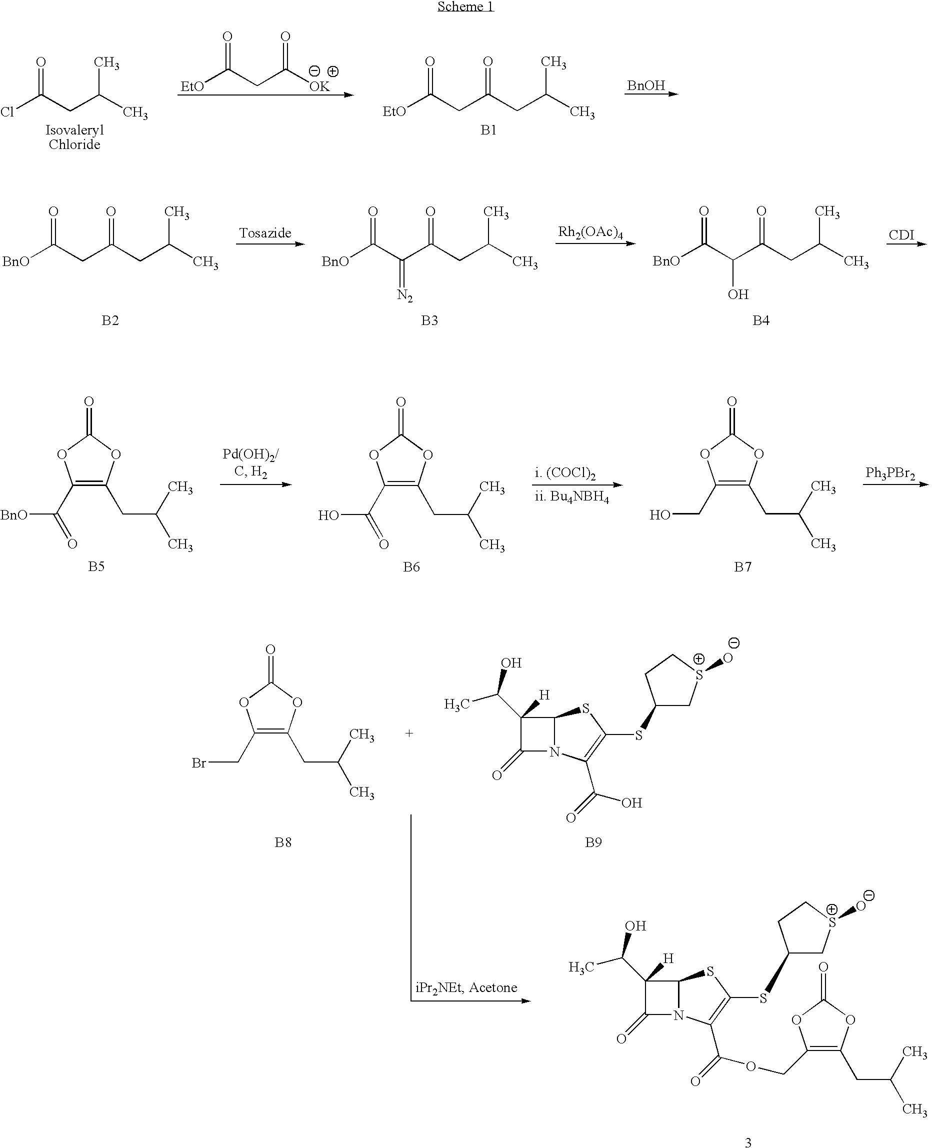

Step 1. Preparation of ethyl 5-methyl-3-oxohexanoate (B1): Potassium ethyl malonate (3680 g, 21.62 mol) in acetonitrile (20 L) was charged to a 50 L three-neck round-bottom flask equipped with a mechanical stirrer, addition funnel, thermometer, nitrogen inlet, drying tube, and cooling bath. The contents of the flask were cooled to 10° C. and treated with triethylamine (2821 mL). Magnesium chloride (2090 g) was then added portion-wise, keeping the temperature below 15° C. The reaction mixture was allowed to warm to about 25° C. and stirred for 2.5 hours. The contents of the flask were cooled to 0° C. and slowly treated with isovaleryl chloride (1303 g, 10.81 mol) while keeping the temperature of the reaction mixture below 5° C. During this time an additional amount of acetonitrile (4000 mL) was added to the resultant viscous mixture. The contents of the flask were then treated with a solution of triethylamine (2000 mL) in acetonitrile (3000 mL) and allowed to stir at 25° C. for 18 hours. The contents of the flask were concentrated at 50° C., and the resultant viscous oil was transferred to a 50 L three-neck round-bottom flask equipped with a mechanical stirrer, addition funnel, thermometer, nitrogen inlet, drying tube, and cooling bath. The contents of the reactor were diluted with toluene (10 L), cooled to 10° C., and treated with hydrochloric acid (15%, 13 L). The contents of the reactor were allowed to warm to 25° C. and mixed for an additional 1 hour. The organic layer was then collected, and the aqueous layer was extracted with toluene (2000 mL). The combined organic layers were then washed with hydrochloric acid (15%, 2×2000 mL), water (2000 mL), and dried over magnesium sulfate. The mixture was then filtered, and the solids washed with toluene (1000 mL). The combined filtrates were then concentrated to provide B1 as a colorless oil. Yield: 1754 g (94%). The product was used in Step 2 below without further purification.

Step 2. Preparation of benzyl 5-methyl-3-oxohexanoate (B2): A solution of B1 (1754 g, 10.18 mol) in benzyl alcohol (1431 g, 13.23 mol) was charged to a 12 L three-neck round-bottom flask equipped with a mechanical stirrer, heating mantle, nitrogen inlet, thermocouple, and short condenser (not cooled). The contents of the flask were then heated at 150° C. for 8 hours. During this time byproduct ethyl alcohol was collected. The reaction mixture was then cooled to provide a benzyl alcohol solution of B2 which was used in Step 3 below without further purification. Yield: 2291 g (product contains approximately 15% of benzyl alcohol). Calculated yield without benzyl alcohol: 1947 g (82%).

Step 3. Preparation of benzyl 2-diazo-5-methyl-3-oxohexanoate (B3): A solution of B2 (1465 g) and tosyl azide (1233 g) in acetonitrile (9,250 mL) was charged to a 22 L three-neck round-bottom flask equipped with a mechanical stirrer, addition funnel, thermometer, nitrogen inlet, drying tube, and cooling bath. The contents of the flask were cooled to 0° C. and slowly treated with triethylamine (740 mL), keeping the reaction temperature below 10° C. The reaction mixture was allowed to warm to 25° C. and stirred at 25° C. for 18 hours. The reaction mixture was then concentrated at 25° C. The resultant oily residue was dissolved in 5000 mL of a 1:2 mixture of heptanes:MTBE and stirred for 30 minutes at 25° C. The resultant suspension was filtered and the solids washed with 1000 mL of a 1:2 mixture of heptanes:MTBE. The combined filtrates were then concentrated, and the resultant residue was purified via silica gel plug (1000 g of silica, H-10″, D-5.5″) eluting with 100% heptanes then gradually 10% AcOEt/heptanes. The eluents containing product were collected and concentrated to provide B3 as a yellow oil. Yield: 1778 g (100%).

Step 4. Preparation of benzyl 2-hydroxy-5-methyl-3-oxohexanoate (B4): A solution of B3 (1627 g) in tetrahydrofuran (13000 mL) was charged to a 50 L three-neck round-bottom flask equipped with a mechanical stirrer, addition funnel, heating mantle, reflux condenser, and thermocouple. The contents of ihe flask were then treated with water (6,000 mL) and rhodium acetate (16 g). The resultant green biphasic mixture was then heated at reflux for about 18 hours. The reaction mixture was concentrated and extracted with ethyl acetate (3×3000 mL). The combined organic layers were with washed with brine, dried over magnesium sulfate, and filtered. The solids were washed with ethyl acetate (500 mL), and the combined filtrates were concentrated. The resultant residue was purified via silica gel plug (1000 g of silica, H-10″, D-5.5°), 50% AcOEt/heptanes, and the eluents containing the product were concentrated to provide B4 as a thick yellow oil. Yield: 1850 g (100%).

Step 5. Preparation of benzyl-5-isobutyl-2-oxo-1,3-dioxole-4-carboxylate (B5): A mixture of B4 (666 g, 2.66 moles), THF (12 L) and diisopropylethylamine (13.3 mL; 9.87 g, 76.4 mmoles) was charged to a 22 L flask equipped with an overhead stirrer, nitrogen inlet, and temperature probe. The contents of the flask were then treated portion-wise over 15 minutes with 1,1′-carbonyldiimidazole (7.98 moles; 1.29 kg) (keeping initial portions less than 100 grams) and stirred at 25° C. for 18 hours. The reaction mixture was concentrated under reduced pressure to remove about 9.5 L of solvent. The resultant slurry was then poured portion-wise into a flask containing a mixture of 2N HCl (5 L) and EtOAc (1 L), keeping the temperature below 20° C. An additional amount of EtOAc (4 L) was added to the flask, and the resultant organic layer was collected and washed with 2N HCl (5 L). The organic phase were passed through a pad of Na₂SO₄ and concentrated under reduced pressure. The resultant residue was then purified via chromatography on silica gel (eluting with an ethyl acetate-heptanes gradient) and the eluents containing compound were concentrated to provide B5 as clear, orange oil that solidified upon standing. Yield: 754 g, ca. 45%.

Step 6. Preparation of 5-isobutyl-2-oxo-1,3-dioxole-4-carboxylic acid (B6): A 2 gallon Parr pressure reactor was charged with Pd(OH)₂ (44.0 g), ethanol (1.20 L) and B5 (440 g). The contents of the reactor were then treated with hydrogen gas (40 psi) at 23° C. for 1.5 hours. During this time, 30.2 g of hydrogen was added to the reactor. The Pd catalyst was removed from reactor, and the reactor contents were treated with additional Pd(OH)₂ (44.0 g). The contents of the reactor were then treated with hydrogen gas (20-40 psi) at 32° C. for 20 minutes. The reactor was vented and purged with nitrogen. The contents of the reactor were then transferred to a carboy vessel (10 L), treated with Celite, and filtered through a 2 L sintered glass funnel. The filter cake was washed with EtOH (2 L), and the combined filtrates were concentrated to provide B6 as a pale yellow solid. Yield: 262 g, 88.4%.

Step 7. Preparation of 4-(hydroxymethyl)-5-isobutyl-1,3-dioxol-2-one (B7): A mixture of B6 (262 g), dichloromethane (3.90 L), and dimethylformamide (10.3 g) was cooled to −0.8° C. and slowly treated with oxalyl chloride (196 g) over a minimum of 30 minutes, keeping the temperature at −1 to 3° C. The reaction mixture was maintained at 0° C. for 30 minutes, warmed to 23° C. over 1 hour, and concentrated to provide the acid chloride intermediate 5-isobutyl-2-oxo-1,3-dioxole-4-carbonyl chloride (B7a) (not shown in Scheme 1). Yield: 289, 100%.

A solution of B7a (287 g) in dichloromethane (3 L) was cooled to −6° C. and treated over 1.3 hours with a mixture of tetrabutylammonium borohydride (98 wt %, 231 g) in dichloromethane (300 mL), keeping the temperature at −5° to 5° C. The reaction mixture was maintained at 0° to 5° C. for 20 minutes and treated with 0.1 M HCl (2.1 L) over 30 minutes, keeping the temperature at 0° to 10° C. The reaction mixture was warmed to 23° C. over 30 minutes and maintained at 23° C. for 1 hour. The resultant organic phase was collected and the aqueous phase washed with dichloromethane. The combined organic phases were washed with brine (1×2.5 L), passed through a 2 L coarse sintered glass funnel containing a plug of Na₂SO₄, and concentrated. The resultant residue was purified via chromatography on silica gel ((eluting with an ethyl acetate-hexanes gradient to provide B7 as a non-viscous clear orange oil. Yield: 149 g, 61.8%.

Step 8. Preparation of 4-(bromomethyl)-5-isobutyl-1,3-dioxol-2-one (B8): A solution of B7 (1.03 mol) in DCM (1845 mL) was cooled to 0° C. and treated with dibromotriphenylphosphorane (463.5 g, 1.1 mol). The mixture was warmed to about 25° C., stirred for 18 hours, and concentrated. The resultant residue was treated with heptane (3600 mL) and filtered, and the solids washed with heptane (3600 mL). The combined filtrates were concentrated, and the resultant brown oil was dissolved in 25% ethyl acetate in heptane (1800 mL). The mixture was then filtered through a bed of silica gel (360.0 g), and the silica was washed with additional 25% ethyl acetate in heptane (1800 mL). The combined filtrates were then concentrated to provide B8 as a brown, viscous oil. Yield: 194 g, 75%. Re-extraction of the residual triphenylphosphine oxide and concentration of the organic layer provided an additional 22 g of 9. Yield (total): 216 g (84%).

Step 9. A mixture of B8 (216 g, 0.92 mol) in acetone (324 mL) was added to a slurry of B9 (sulopenem) (324 g, 0.93 mol) (prepared according to Example 11 of U.S. Pat. No. 5,013,729) in acetone (2592 mL). The resultant mixture was then treated with diisopropylethylamine (125.3 g, 0.97 mol) in acetone (324 mL) and stirred at 25° C. for 9 hours. The mixture was then treated with water (1123 mL) and heptane (1123 mL), and the resultant aqueous phase was further extracted with heptane (1123 mL). The aqueous phase was then distilled under reduced pressure to remove acetone followed by extraction with ethyl acetate (3×1123 mL). The combined ethyl acetate extracts were washed with aqueous sodium thiosulfate (1447 mL, 0.31 Molar), water (1447 mL) and brine (1447 mL). The organic phase was then treated with activated carbon (65 g) and celite (65 g). The filter cake was washed ethyl acetate (2×648 mL), and the combined filtrates were concentrated. The resultant residue was then treated with ethyl acetate (243 mL), and the resultant suspension was heated to about 70° C. The resultant solution was then treated with tert-butyl methyl ether (486 mL) and cooled until precipitation occurred. The resultant suspension was granulated at a temperature of −5° C. to 15° C. for a minimum of 1 hour. The solids were collected, washed with tert-butyl methyl ether (2×486 mL), and dried to provide 3 as a pale yellow solid. Yield: 320 g (69%).

¹H NMR (400 MHz, CDCl₃) δ 5.69 (d, J=1.4 Hz, 1H), 4.99 (d, J=13.9 Hz, 1H), 4.91 (d, J=13.9 Hz, 1H), 4.4-4.1 (m, 1H), 3.9-3.85 (m, 1H), 3.8-3.65 (m, 2H), 3.15-3.1 (m, 1H), 2.9-2.6 (m, 4H), 2.37 (d, J=7.0 Hz, 2H), 1.97-1.90 (m, 1H), 1.31 (d, J=6.3 Hz, 3H), 0.95-0.93 (m, 6H) ppm.

¹³C NMR (100 MHz, CDCl₃) δ 21.36, 21.94, 21.99, 26.42, 32.34, 33.29, 46.73, 52.58, 54.04, 61.01, 65.02, 65.02, 65.28, 71.69, 117.15, 133.90, 143.16, 152.24, 153.9, 158.89, 172.07 ppm.

### Example 2

Preparation of (5-ethyl-2-oxo-1,3-dioxol-4-yl)methyl (5R,6S)-6-[(1R)-1-hydroxyethyl]-7-oxo-3-[[(1R,3S)-tetrahydro-1-oxido-3-thienyl]thio]-4-thia-1-azabicyclo[3.2.0]hept-2-ene-2-carboxylate (1): Compound 1 was prepared in a manner similar to that described above for making 3, except that 4-(bromomethyl)-5-ethyl-1,3-dioxol-2-one was used instead of 4-(bromomethyl)-5-isobutyl-1,3-dioxol-2-one.

### Example 3

Preparation of (5-propyl-2-oxo-1,3-dioxol-4-yl)methyl (5R,6S)-6-[(1R)-1-hydroxyethyl]-7-oxo-3-[[(1R,3S)-tetrahydro-1-oxido-3-thienyl]thio]-4-thia-1-azabicyclo[3.2.0]hept-2-ene-2-carboxylate (2): Compound 2 was prepared in a manner similar to that described above for making 3, except 4-(bromomethyl)-5-propyl-1,3-dioxol-2-one was used instead of 4-(bromomethyl)-5-isobutyl-1,3-dioxol-2-one.

The hemihydrate form of compound 2 was prepared by dissolving 2 (13.8 mg) in 1-propanol (300 μL) and allowing the 1-propanol to slowly evaporate to provide the hemihydrate as clear, colorless crystals.

### Comparative Example 4

Preparation of (5-Methyl-2-oxo-1,3-dioxol-4-yl)methyl (5R,6S)-6-[(1R)-1-hydroxyethyl]-7-oxo-3-[[(1R,3S)-tetrahydro-1-oxido-3-thienyl]thio]-4-thia-1-azabicyclo[3.2.0]hept-2-ene-2-carboxylate (C1):

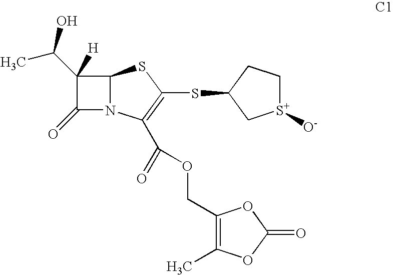

Comparative compound C1 was prepared according to the method described in U.S. Pat. No. 5,013,729.

Biological, melting point and solubility data for the sulopenem prodrugs 1 to 3 are shown in Table 2. Also shown in Table 2 are data for comparative compound C1 (the methyl analog of the sulopenem prodrugs of the invention).

TABLE 2
Biological data for sulopenem prodrugs.

| | | | | S9 | Whole | Caco-2 | | | |
|---|---|---|---|---|---|---|---|---|---|
| | HIJ CLint | HIJ Km | S9 Conv | Half-life | Blood | Permeability | | Melt. Pt. | Solubility |
| Cpd. | (mL/min) | (μM) | (%) | (min) | Conv (%) | (cm/sec) | clogP* | (° C.) | (ug/mL) |
| C1 | 0.13 | 172 | 90.3 | 2.10 | 77 | 5.00 × 10⁻⁷ | −1.98 | 159-160 | 453 |
| 1 | 0.14 | 120 | 95 | 3.11 | 75 | 2.00 × 10⁻⁶ | −1.45 | 136 | 1200 |
| 2 | 0.16 | 22 | 83 | 3.62 | 67 | 4.1 × 10⁻⁶ | −0.92 | 119-122** | 507 |
| 3 | 0.16 | 41 | 67 | 6.09 | 84 | 5.02 × 10⁻⁶ | −0.52 | 130-132 | 809 |

*Calculated using ClogP v4.3 from Biobyte Corporation (available at www.biobyte.com).
**The hemihydrate form of compound 2 was used, and it displayed a broad low temperature endotherm attributed to dehydration in the 25° C.-65° C. region. The second endotherm (reported in Table 2) is the melting of the dehydrated hemihydrate.

The aqueous solubility of the sulopenem prodrugs of the invention was determined in phosphate buffer (pH 5), which simulates physiological conditions. The results in Table 2 show that the sulopenem prodrugs of the invention are more soluble in the phosphate buffer at ambient temperature than comparative compound C1. This result is surprising, because replacement of the methyl group of C1 with ethyl (compound 1), propyl (compound 2), or isobutyl (compound 3) groups would expectedly reduce aqueous solubility. (See, e.g., C. Goosen et al., Pharmaceutical Research 19 (2002) 13-19; and D. Y. Hung et al., Int. J. Pharmaceutics 153 (1997) 25-39.)

The results (Table 2) also show that the sulopenem prodrugs of the invention have a higher Caco-2 permeability than comparative compound C1. Compared to C1, compound 1 exhibits about a 4-fold increase in permeability, compound 2 exhibits about an 8-fold increase in permeability, and compound 3 exhibits about a 10-fold increase in permeability. An increase in Caco-2 permeability with increasing alkyl chain length is not unexpected due to increased lipophilicity. However, the magnitude of the increase of Caco2-permeability of compounds 1 to 3 relative to C1 is surprising for compounds in this range of molecular weight (G. Camenisch et al. Eur. J. Pharm. Sci. 6 (1998) 313-319).

What is claimed is:

1. A compound of formula (I): 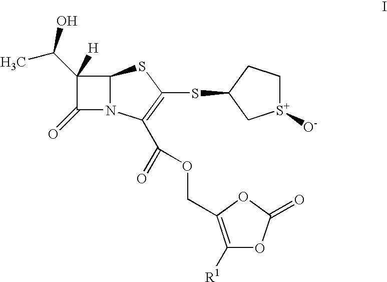
wherein R¹ is —(CH₂C₈)alkyl, or a solvate or hydrate thereof.

2. The compound of claim 1 wherein R¹ is —CH₂CH₃, or a solvate or hydrate thereof.

3. The compound of claim 1 wherein R¹ is —CH₂CH₂CH₃, or a solvate or hydrate thereof.

4. The compound of claim 1 wherein R¹ is —CH₂CH(CH₃)₂, or a solvate or hydrate thereof.

5. A composition comprising the compound of claim 1, or a solvate or hydrate thereof, and one or more additional ingredients.

6. The composition of claim 5 wherein said one or more additional ingredients is an active agent.

7. The composition of claim 5 wherein said compound of claim 1, or a solvate or hydrate thereof, is present in an amount from about 200 mg to about 4000 mg.

8. The composition of claim 5 wherein R¹ is —CH₂CH(CH₃)₂.

9. A method of treating a bacterial infection comprising administering a therapeutically effective amount of the compound of claim 1, or a solvate or hydrate thereof, to a patient in need thereof.

10. The method of claim 9 wherein R¹ is —CH₂CH(CH₃)₂.

11. The method of claim 9, wherein said compound, or a solvate or hydrate thereof, is administered in an amount of from about 400 mg to about 6000 mg over a 24 hour period.

12. The method of claim 9, wherein said compound of claim 1, or a solvate or hydrate thereof, is administered orally.

13. The method of claim 9, wherein said bacterial infection is selected from the group consisting of acute exacerbation of chronic bronchitis, sinusitis, otitis media, brain abscess, pharyngitis, meningitis, uncomplicated/complicated urinary tract infections, pyelonephritis, hospital-acquired pneumonia, community-acquired pneumonia, surgical prophylaxis, uncomplicated/complicated skin and skin structure infections, intra-abdominal infections, prostatitis, obstetric and gynecological infections, bone and joint infections, diabetic foot, and bacteremia.

14. The method of claim 9, wherein said bacterial infection is a gram-positive infection.

15. The method of 9, wherein said bacterial infection is a gram-negative infection except for Pseudomonas aeruginosa, Acinetobacter spp. and Stenotrophomonas maltophilia.

16. The method of claim 9 further comprising one or more additional active agents.

17. The method of claim 16, wherein said one or more additional active agents is an antibacterial agent.

19. The compound (5-propyl-2-oxo-1,3-dioxol-4-yl)methyl (5R,6S)-6-[(1R)-1-hydroxyethyl]-7-oxo-3-[[(1R,3S)-tetrahydro-1-oxido-3-thienyl]thio]-4-thia-1-azabicyclo[3.2.0]hept-2-ene-2-carboxylate.

20. The compound (5-(3-methylpropyl)-2-oxo-1,3-dioxol-4-yl)methyl (5R,6S)-6-[(1R)-1-hydroxyethyl]-7-oxo-3-[[(1R,3S)-tetrahydro-1-oxido-3-thienyl]thio]-4-thia-1-azabicyclo[3.2.0]hept-2-ene-2-carboxylate.
